(19) United States
(12) Patent Application Publication
(10) Pub. No.: US 2017/0266391 A1
(43) Pub. Date: Sep. 21, 2017

(54) INJECTION NEEDLE DEVICE FOR ENDOSCOPE

(71) Applicant: KANEKA CORPORATION, Osaka-shi, Osaka (JP)

(72) Inventor: Masato TSUEDA, Okaya-shi, Nagano (JP)

(73) Assignee: KANEKA CORPORATION, Osaka-shi, Osaka (JP)

(21) Appl. No.: 15/504,521

(22) PCT Filed: Aug. 27, 2015
(86) PCT No.: PCT/JP2015/074308

§ 371 Date: Feb. 16, 2017

(30) Foreign Application Priority Data
Sep. 22, 2014 (JP) 2014-192891

(51) Int. Cl. A61M 5/32; A61L 31/04; A61L 31/06; A61B 1/018

(52) U.S. Cl. CPC A61M 5/329; A61B 1/018; A61M 5/3287; A61L 31/048; A61L 31/06

(57) ABSTRACT

The present invention provides an injection needle device for an endoscope that can prevent an injection needle from unintentionally being exposed. An injection needle device for an endoscope comprises an outer tubular body (20), an inner tubular body (30) provided in the outer tubular body (20) and an injection needle (50) inserted into one end part of the inner tubular body (30), wherein the inner tubular body (30) includes an inner layer (31) and an outer layer (35) directly or indirectly contacted with an outer surface of the inner layer (31), and a water absorption coefficient of the outer layer (35) is lower than that of the inner layer (31).

### TECHNICAL FIELD

The present invention relates to an injection needle device for endoscope for injecting drug solution into a tissue site in a body cavity of a human body through a forceps channel of an endoscope.

### BACKGROUND ART

An endoscopic surgery is performed, while the state in the body cavity is observed by inserting an endoscope mainly from the mouth, nose, or anus of a patient. In the injection into the body cavity in the endoscopic surgery, an injection needle device for endoscope is inserted into the body cavity of the patient through a forceps channel of the endoscope from a forceps opening of the endoscope, and an injection needle of this injection needle device is inserted into a tissue site of the patient to be treated to inject drug solution thereinto. The injection needle device for endoscope includes an inner tubular body which is for delivering drug solution and has the injection needle provided thereto, and an outer tubular body that stores the injection needle and the inner tubular body. When the inner tubular body moves relative to the outer tubular body, friction occurs on the contact surface between the inner tubular body and the outer tubular body, which disturbs the surgery. To prevent such friction, a polymer material having high slidability, such as polypropylene (PP), polytetrafluoroethylene (PTFE), or nylon, is used for the inner tubular body and the outer tubular body.

For example, Patent Literature 1 discloses an injection needle device for endoscope including: a mantle tube formed from a flexible synthetic resin tube; a solution delivery tube which is inserted into the mantle tube so as to freely pass therethrough and formed from a flexible and elastic synthetic resin tube; a needle part formed by diagonally cutting the tip of the solution delivery tube; and a solution injection opening provided on the base end side of the solution delivery tube for feeding solution into the solution delivery tube. Patent Literature 1 also discloses using low-density polyethylene, high-density polyethylene, tetrafluoroethylene-perfluoroalkyl vinyl ether copolymer (PFA), polytetrafluoroethylene (PTFE), and the like as the material of the mantle tube of the injection needle device for endoscope, and using nylon, polypropylene, polyimide resin, and the like as the material of the solution delivery tube.

### CITATION LIST

### Patent Literature

### Patent Literature 1

Japanese Unexamined Patent Application Publication No. H08-336591

### SUMMARY OF THE INVENTION

### Problems to be Solved by the Invention

However, if nylon is used as the material of the solution delivery tube as in the injection needle device for endoscope disclosed in Patent Literature 1, a needle part may be exposed from a mantle tube for some reason before an injection needle device for endoscope is actually used.

In view of this, the present invention aims to provide an injection needle device for endoscope that can prevent an injection needle from unintentionally being exposed.

### Solution to the Problems

To address the foregoing problem, the present inventor has studied the relationship between an environment where an injection needle device for endoscope is placed and a material used for this injection needle device. An injection needle device for endoscope may be subjected to a sterilization process using high-pressure steam or the like during the manufacturing process thereof in consideration of safety, or may be stored for a long time in a high humidity environment, such as in a warehouse of a manufacturer, after manufacture. The present inventor has considered that moisture resistance is one of important factors for considering the material of a tubular body in the light of the aspect in which, in such a high humidity environment, a material having a high water absorption coefficient expands to cause variation in size and is extended longer than the original design. The component that should be particularly considered in the light of particularly moisture resistance is a tubular body that is used for delivering drug solution and is provided with an injection needle on one end thereof.

The material for the tubular body is selected through comprehensive evaluation of sliding performance, torsional deformation resistance (kink resistance), chemical resistance, excellent processability, bondability to other members, cost, and the like, in addition to moisture resistance. Among others, nylon is often used due to merits of being excellent in sliding performance and bondability to other member and having low production cost. However, nylon has a high water absorption coefficient, so that, if being exposed to the high humidity environment described above, it is likely to expand to cause variation in size. On the other hand, a tubular body composed of fluorine resin such as PTFE or PFA or polyethylene as the material thereof has excellent sliding performance and torsional deformation resistance, and further, is stable in size due to a low water absorption coefficient. However, fluorine resin or polyethylene resin needs to be subjected to a chemical process or polishing process on the surface thereof for enhancing bondability to other member, which is likely to lead to a complex manufacturing process or increase in cost. In view of this, as a result of the extensive studies, the present inventor has found that a tubular body which hardly varies in size even under a high humidity environment can be obtained, if a tubular body provided inside is configured to have a two-layer structure, and materials having different water absorption coefficients are used for each layer. Thus, the present inventor has achieved the present invention.

That is, the injection needle device for endoscope of the present invention comprises an outer tubular body, an inner tubular body provided in the outer tubular body and an injection needle inserted into one end part of the inner tubular body, wherein the inner tubular body includes an inner layer and an outer layer directly or indirectly contacted with an outer surface of the inner layer, and a water absorption coefficient of the outer layer is lower than that of the inner layer. The injection needle device for endoscope according to the present invention is configured to have a two-layer structure in which the inner layer is formed on the outer layer of the inner tubular body and to allow drug solution to be in contact with the inner surface of the inner layer, whereby the outer layer can be protected from drug solution. In addition, the outer layer in the present invention has a low water absorption coefficient. Therefore, even under a high humidity environment, the inner tubular body is less likely to expand in the axial direction, whereby the unintentional exposure of the injection needle from the outer tubular body can be prevented. Furthermore, the inner tubular body is less likely to expand also in the radial direction, which can prevent the inner tubular body from being in contact with the outer tubular body to deteriorate sliding performance.

In the inner tubular body according to the present invention, it is also preferred that the water absorption coefficient of the outer layer is 0.04 times or less of that of the inner layer. When the water absorption coefficients of the outer layer and the inner layer of the inner tubular body are set in this manner, the expansion of the inner tubular body under a high humidity environment can be suppressed.

In the inner tubular body according to the present invention, it is also preferred that a thickness of the outer layer is 2.5 times or more of that of the inner layer. The thicker the outer layer having a low water absorption coefficient is, the more the expansion of the inner tubular body under a high humidity environment can be suppressed.

In the injection needle device for endoscope of the present invention, it is also preferred that a length in an axial direction of the inner tubular body after being left under the following high humidity environment is 1.013 times or less of that before being left under the following high humidity environment, when the inner tubular body is left for three hours under an environment of relative humidity of 40% to 60% and subsequently left for three hours under a high humidity environment of relative humidity of 80% or more. If the difference in length in the axial direction of the inner tubular body is small before and after the inner tubular body is left under a high humidity environment, the exposure of the injection needle from the outer tubular body can be prevented under the high humidity environment.

In the inner tubular body according to the present invention, it is also preferred that a contact angle of the inner layer is smaller than that of the outer layer. The smaller the contact angle of the inner layer is, the higher the adhesion force to the other member is, and thus, the adhesion to the injection needle or the like can be enhanced, for example.

In the inner tubular body according to the present invention, it is also preferred that the inner layer is composed of nylon, and the outer layer is composed of polyethylene. Among polymer materials, nylon has a small contact angle and easy to be bonded to the injection needle. In addition, due to a low water absorption coefficient, polyethylene is less likely to expand even if it is left under a high humidity environment, thereby being capable of stabilizing the dimension of the inner tubular body.

In the present invention, it is also preferred that the injection needle and the inner layer are connected with each other by thermocompression bonding. Since the inner layer and the injection needle can reliably be fixed to each other, the injection needle can be prevented from falling off from the inner layer.

The present invention includes the injection needle device for endoscope further comprising a first handle part connected to the other end of the inner tubular body and capable of moving in the axial direction of the inner tubular body. When the first handle part is moved in the axial direction, the inner tubular body is also moved along with the first handle part to allow the injection needle to be exposed from the outer tubular body, whereby an operation of injecting drug solution is enabled.

The present invention includes the injection needle device for endoscope further comprising a second handle part connected to the outer tubular body and provided with a fixing means that secures the first handle part. When the first handle part and the second handle part are secured, the position of the injection needle in the axial direction of the inner tubular body is fixed, so that the operation of injecting drug solution is enabled with stability.

In the present invention, it is also preferred that an inner diameter at an end part of the injection needle side of the outer tubular body is smaller than an outer diameter of the inner tubular body. When the inner diameter at the end part of the injection needle side of the outer tubular body is set smaller, the inner tubular body is stuck at a reducing diameter part when the injection needle is exposed from the outer tubular body for injection of drug solution. Thus, the exposure of the inner tubular body from the outer tubular body can be prevented.

In the present invention, it is also preferred that a length of the inner tubular body is longer than that of the outer tubular body in the axial direction of the inner tubular body. Since the length of the inner tubular body inserted into the first handle part is larger, the first handle part can more stably hold the inner tubular body.

In the present invention, it is also preferred that the outer tubular body is composed of polytetrafluoroethylene, and the outer layer of the inner tubular body is composed of a material other than polytetrafluoroethylene. Polytetrafluoroethylene (PTFE) has a small friction coefficient. Therefore, even when a material other than PTFE is used for the material of the outer layer of the inner tubular body, high sliding performance can be obtained.

### Advantageous Effects of Invention

The injection needle device for endoscope according to the present invention is configured to have a two-layer structure in which the inner layer is formed on the outer layer of the inner tubular body and to allow drug solution to be in contact with an inner surface of the inner layer, whereby the outer layer can be protected from drug solution. In addition, the outer layer in the present invention has a low water absorption coefficient. Therefore, even under a high humidity environment, the inner tubular body is less likely to expand in the axial direction, whereby the unintentional exposure of the injection needle from the outer tubular body can be prevented. Furthermore, the inner tubular body is less likely to expand also in the radial direction, which can prevent from being in contact with the outer tubular body to deteriorate sliding performance.

### BRIEF DESCRIPTION OF THE DRAWINGS

FIG. 1 shows a plan view (a partial cross-sectional view) of an injection needle device for endoscope in accordance with an embodiment of the present invention.

FIG. 2 shows a cross-sectional view taken along an axial direction when the injection needle is stored in an outer tubular body of an injection needle device for endoscope in accordance with the embodiment of the present invention.

FIG. 3 shows a cross-sectional view taken along an axial direction when the injection needle is exposed from an outer tubular body of an injection needle device for endoscope in accordance with the embodiment of the present invention.

### MODE FOR CARRYING OUT THE INVENTION

The injection needle device for endoscope of the present invention comprises an outer tubular body, an inner tubular body provided in the outer tubular body and an injection needle inserted into one end part of the inner tubular body, wherein the inner tubular body includes an inner layer and an outer layer directly or indirectly contacted with an outer surface of the inner layer, and a water absorption coefficient of the outer layer is lower than that of the inner layer. The injection needle device for endoscope according to the present invention is configured to have a two-layer structure in which the inner layer is formed on the outer layer of the inner tubular body and to allow drug solution to be in contact with the inner surface of the inner layer, whereby the outer layer can be protected from drug solution. In addition, the outer layer in the present invention has a low water absorption coefficient. Therefore, even under a high humidity environment, the inner tubular body is less likely to expand in the axial direction, whereby the unintentional exposure of the injection needle from the outer tubular body can be prevented. Furthermore, the inner tubular body is less likely to expand also in the radial direction, which can prevent the inner tubular body from being in contact with the outer tubular body to deteriorate sliding performance.

The injection needle device for endoscope is a treatment tool used for injecting drug solution into a tissue site in a body cavity in an endoscopic surgery, and it is inserted into the body cavity through a forceps opening of an endoscope, for example. The injection needle device for endoscope according to the present invention includes an outer tubular body, an inner tubular body provided in the outer tubular body and an injection needle inserted into one end part of the inner tubular body.

The outer tubular body is a tubular body for storing the injection needle inside so as to prevent a tissue site, which is not a subject to be treated, in the body cavity or the interior of the forceps channel of the endoscope from being damaged. The inner tubular body into which the injection needle is inserted is disposed in the outer tubular body.

The inner tubular body is a tubular body whose a lumen is a flow path for flowing drug solution, the one end part of the inner tubular body, that is one end part of the flow path, is inserted the injection needle. Drug solution is injected by inserting the injection needle into the tissue site to be treated in the body cavity of a patient. A length in an axial direction of the inner tubular body may be set in consideration of the distance between the forceps opening of the endoscope and the tissue site into which the injection needle is to be inserted, or the like, and for example, it can be set to be 2500 mm or 1500 mm.

The injection needle is inserted into the one end part of the inner tubular body and an outer surface of the injection needle directly or indirectly is contacted with an inner surface of the inner tubular body. In order to reliably be fixed the inner layer and the injection needle and to be prevented the injection needle from falling off from the inner layer, it is preferred that the injection needle and the inner layer are connected with each other by thermocompression bonding. In addition, the injection needle and the inner layer are preferably bonded to each other by an adhesive. Note that the type of the adhesive is not particularly limited. Examples of usable adhesives include polyurethane-based adhesive, epoxy-based adhesive, cyano-based adhesive, and silicon-based adhesive.

The inner tubular body has the two-layer structure having the inner layer and the outer layer directly or indirectly contacted with the outer surface of the inner layer. It is configured such that an inner surface of the inner layer is directly contacted with drug solution, and therefore, the outer layer is basically not directly contacted with drug solution. Specifically, the outer layer is protected by the inner layer, whereby the outer layer can be protected from being immersed into drug solution.

The outer surface of the inner layer and an inner surface of the outer layer are directly or indirectly contacted with each other. Here, the state of being directly contacted indicates that the inner layer and the outer layer are bonded to each other by thermocompression bonding or fusion bonding, for example. In addition, the state of being indirectly contacted indicates that the inner layer and the outer layer are bonded to each other through an adhesion layer formed from an adhesive, for example. Note that the type of the adhesive used for the bonding between the outer layer and the inner layer is not particularly limited. Examples of usable adhesives include polyurethane-based adhesive, epoxy-based adhesive, cyano-based adhesive, and silicon-based adhesive.

In the present invention, it is also preferred that the inner layer of the inner tubular body is provided on only a part of the outer layer in the axial direction. For example, a part, which is bonded to the injection needle, of the inner tubular body may have a two-layer structure of the inner layer and the outer layer, and a part, which is not bonded to the injection needle, of the inner tubular body has only the outer layer.

In addition, in the present invention, a water absorption coefficient of the outer layer is lower than that of the inner layer. The water absorption coefficient is a rate of change of mass (unit: %) before and after the immersion in water at 23° C. for 24 hours, and measured in accordance with ASTM D570 of ASTM test method. Table 1 shows water absorption coefficients of high-density polyethylene (HDPE), polypropylene (PP), nylon, low-density polyethylene (LDPE), tetrafluoroethylene-perfluoroalkyl vinyl ether copolymer (PFA), polyether ether ketone (PEEK), polytetrafluoroethylene (PTFE), ethylene-tetrafluoroethylene copolymer (ETFE), measured in accordance with ASTM D570.

TABLE 1

| Material | Water absorption coefficient (%) |
|---|---|
| High-density polyethylene (HDPE) | 0.01 |
| Polypropylene (PP) | 0.01 |
| Nylon | 0.9 |
| Low-density polyethylene (LDPE) | 0.02 |
| Tetrafluoroethylene-perfluoroalkyl | 0.03 |
| vinyl ether copolymer (PFA) | |
| Polyether ether ketone (PEEK) | 0.2 |
| Polytetrafluoroethylene (PTFE) | 0.001 |
| Ethylene-tetrafluoroethylene | 0.02 |
| copolymer (ETFE) | |

When absorbing water, the inner tubular body expands in the axial direction and radial direction of the inner tubular body in proportion to the water absorption coefficient. In view of this, the lower the water absorption coefficient is, the more the expansion of the inner tubular body can be suppressed.

In the present invention, the outer layer having a low water absorption coefficient is bonded on the outer surface of the inner layer having a high water absorption coefficient. Therefore, even if the inner layer expands by absorbing water, the expansion of the inner layer is suppressed by the outer layer, by which the entire of the inner tubular body hardly expands. This configuration can prevent the exposure of the injection needle from the outer tubular body caused by the expansion of the inner tubular body in the axial direction, or deterioration in sliding performance between the inner tubular body and the outer tubular body caused by the expansion of the inner tubular body in the radial direction, under a high humidity environment.

In the present invention, it is also preferred that the water absorption coefficient of the outer layer is 0.04 times or less of that of the inner layer. The larger the difference between the water absorption coefficient of the outer layer and the water absorption coefficient of the inner layer is, the more the expansion of the entire inner tubular body is controlled mainly by the expansion of the outer layer. Therefore, the expansion of the inner tubular body can be suppressed even under a high humidity environment. Accordingly, the water absorption coefficient of the outer layer is more preferably 0.025 times or less of that of the inner layer, even more preferably 0.02 times or less, particularly preferably 0.015 times or less, most preferably 0.01 times or less.

In the present invention, it is also preferred that a thickness of the outer layer is 2.5 times or more of that of the inner layer. The larger the thickness of the outer layer having a low water absorption coefficient is, the more the expansion of the entire inner tubular body is controlled by the expansion of the outer layer. Therefore, the expansion of the inner tubular body can be suppressed even under a high humidity environment. Accordingly, the thickness of the outer layer is more preferably 3 times or more of that of the inner layer, even more preferably 4 times or more, even still more preferably 5 times or more, particularly preferably 7 times or more, most preferably 10 times or more.

The injection needle device for endoscope is set such that, under an environment having relative humidity of 40% to 60%, the tip of the injection needle is located on the position distant from the inside of the end part of the outer tubular body by about 5 mm to 20 mm. This is mainly because, even if the inner tubular body expands when the injection needle device for endoscope is left under a high humidity environment, the insertion depth (stroke width) of the injection needle device in the operation of injecting drug solution is set as appropriate, while the exposure of the injection needle from the outer tubular body is prevented. For example, if the inner tubular body having a length in the axial direction of about 2500 mm expands and the length in the axial direction is increased by 1% (that is, 25 mm in this case), the injection needle is likely to be exposed from the outer tubular body. Therefore, the dimensions of the outer tubular body and the inner tubular body need to be set strictly. Accordingly, it is preferred that the dimensions of the outer tubular body and the inner tubular body are changed as little as possible even if the ambient environment is changed.

Thus, in the injection needle device for endoscope of the present invention, it is preferred that a length in the axial direction of the inner tubular body after being left under the following high humidity environment is 1.013 times or less of that before being left under the following high humidity environment, when the inner tubular body is left for three hours under an environment of relative humidity of 40% to 60% and subsequently left for three hours under a high humidity environment of relative humidity of 80% or more. The smaller the difference in length in the axial direction of the inner tubular body is before and after the inner tubular body is left under a high humidity environment, the less likely the inner tubular body expands in the axial direction, whereby the exposure of the injection needle from the outer tubular body can be prevented. In addition, the length in the axial direction of the inner tubular body after being left under the high humidity environment is more preferably 1.010 times or less of that before being left under the high humidity environment, even more preferably 1.008 times or less, even still more preferably 1.006 times or less, even further more preferably 1.005 times or less, particularly preferably 1.003 times or less, most preferably 1.001 times or less. Furthermore, the lower limit of the length in the axial direction of the inner tubular body after the inner tubular body is left under a high humidity environment with respect to the length in the axial direction of the inner tubular body before the inner tubular body is left under the high humidity environment is not particularly limited, it may be 1.0 times or more, for example.

As described above, as the condition for measuring the ratio of the length in the axial direction of the inner tubular body after being left under a high humidity environment to the length in the axial direction of the inner tubular body before being left under the high humidity environment, the inner tubular body, which has been left for 3 hours under the high humidity environment having relative humidity of 40% to 60%, is left for 3 hours under a high humidity environment having relative humidity of 80% or more. However, in the case in which it is apparent that the expansion/contraction of the inner tubular body is sufficiently stabilized (for example, the expansion/contraction amount exceeds 80% of the saturation expansion/contraction amount), the time for leaving the inner tubular body can be appropriately changed to 30 minutes, 1 hour, 1.5 hours, 2 hours, or the like.

To reliably fix the injection needle to the inner tubular body, the inner layer preferably has high adhesion. It is necessary that the intermolecular distances of a solid and liquid are made close to each other to bond the solid and liquid to each other, and high wettability on the surface of the solid is one of the methods. Wettability is found by measuring the contact angle θ of a water droplet, and a contact angle is an angle made by a surface of a solid and a tangent drawn from the part, on which the solid, liquid, and gas are contacted with one another, toward the curved surface of the liquid. And the contact angle can be measured in accordance with the method specified in JIS R 3257. When the contact angle is large and obtuse, wettability is small, so that adhesion is low. On the other hand, when the contact angle is small and acute, wettability is large, so that adhesion is high. Therefore, it is also preferred that the contact angle of the inner layer is smaller than that of the outer layer in order to reliably bond the inner layer and the injection needle to each other and to prevent the injection needle from falling off from the inner layer. The evaluation of adhesion force based on the contact angle is effective for the case of using a water-based adhesive.

In evaluating adhesion force between the material of the inner layer and an adhesive or the like including adhesives other than the water-based adhesive, it is also preferred to use surface free energy γS (unit: N/m), which represents intermolecular force of a solid surface as a numerical value, as an index of wettability. The surface free energy can be obtained from the following extended Fowkes' equation (1) and Young's equation (2) using a contact angle and a value of surface tension of measurement liquid. Here, the measurement liquid of the contact angle can be selected from pure water, liquid paraffin, glycerin, methylene iodide, n-hexadecane, α-bromonaphthalene and the like.

γL(1+cos θ)/2=(γSd×γLd)^{1/2}+(γSp×γLp)^{1/2}+(γSh×γLh)^{1/2} (1)

γS=γSd+γSp+γSh (2)

γL: a surface tension of the measurement liquid
γLd: a dispersed component of the surface tension of the measurement liquid
γLp: a polar component of the surface tension of the measurement liquid
γLh: a hydrogen-bond component of the surface tension of the measurement liquid
γS: a surface free energy
γSd: a dispersed component of the surface free energy
γSp: a polar component of the surface free energy
γSh: a hydrogen-bond component of the surface free energy

The larger the surface free energy is, the higher the adhesion force becomes. Accordingly, it is also preferred that the surface free energy of the inner layer is larger than that of the outer layer.

In the present invention, it is also preferred that an inner diameter at an end part of the injection needle side of the outer tubular body is smaller than an outer diameter of the inner tubular body. When the inner diameter at the end part of the injection needle side of the outer tubular body is set smaller, the inner tubular body is stuck at this reducing diameter part when the injection needle is exposed from the outer tubular body for injection of drug solution. Thus, the exposure of the inner tubular body from the outer tubular body can be prevented.

In order to prevent deterioration in sliding performance between the outer tubular body and the inner tubular body, it is preferred that lumens of the outer tubular body and the inner tubular body have nearly true circle shape or have low ellipticity. For example, preferably, the ellipticities of the outer tubular body and the inner tubular body are 10% or less, more preferably 8% or less, and even more preferably 5% or less. The ellipticity (%) of the outer tubular body and the ellipticity (%) of the inner tubular body can be obtained from the equations below.

The ellipticity (%) of the outer tubular body=(the outer diameter of the outer tubular body−the inner tubular diameter of the outer tubular body)/the inner diameter of the outer tubular body×100

The ellipticity (%) of the inner tubular body=(the outer diameter of the inner tubular body−the inner diameter of the inner tubular body)/the inner diameter of the inner tubular body×100

In addition, the ellipticity of the outer tubular body is also preferably equal to the ellipticity of the inner tubular body or less. For example, an absolute value of difference between the ellipticity of the outer tubular body and the ellipticity of the inner tubular body is preferably 5% or less, more preferably 4% or less, even more preferably 2% or less. Furthermore, a lower limit of the absolute value of difference between the ellipticity of the outer tubular body and the ellipticity of the inner tubular body is not restricted but it may, for example, 1% or more.

In an axial direction of a tubular body of the present invention, it is also preferred that a length of the inner tubular body is longer than that of the outer tubular body. Since the length of the inner tubular body inserted into the first handle part becomes large, the first handle part can more stably hold the inner tubular body.

Since the outer tubular body and the inner tubular body are bent into various shapes in the body cavity of the patient, it is preferred that the outer tubular body and the inner tubular body have flexibility, and it is also preferred that the outer tubular body and the inner tubular body have high slidability. Thus, it is preferred that the outer tubular body and the inner tubular body are composed of polymer materials such as polypropylene (PP), polytetrafluoroethylene (PTFE), ethylene-tetrafluoroethylene copolymer (ETFE), nylon, low-density polyethylene (LDPE), high-density polyethylene (HDPE), tetrafluoroethylene-perfluoroalkyl vinyl ether copolymer (PFA), polyimide (PI), polyether ether ketone (PEEK) and the like.

It is also particularly preferred that the inner layer of the inner tubular body is composed of nylon, and the outer layer of that is composed of polyethylene. Nylon which has a small contact angle is easy to be bonded to the injection needle, and polyethylene which has a water absorption coefficient lower than that of nylon can prevent expansion of the inner tubular body under a high humidity environment.

When the outer tubular body is composed of PTFE, sliding performance between the outer tubular body and the inner tubular body is enhanced due to a small friction coefficient of PTFE. Notably, the study made by the present inventor shows that, if PTFE is used as the material of the outer tubular body, a material other than PTFE 15 preferably used for the outer layer of the inner tubular body from the viewpoint of sliding performance.

It is also preferred that a roughening process is performed on the outer surface of the injection needle to enhance adhesion between the outer surface of the injection needle and the inner surface of the inner tubular body. For example, the surface roughness Ra of the injection needle can be set to be 1.0 μm or more. Although the material of the injection needle is not particularly limited, a metal material such as stainless or Ni—Ti alloy or a polymer material such as polyethylene can be used.

Also, a known hollow injection needle can be used for the injection needle. The tip of the injection needle, that is, the side which is first in contact with the tissue site of the patient, can be diagonally cut at an angle of 10 degrees to 40 degrees.

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

### Embodiment

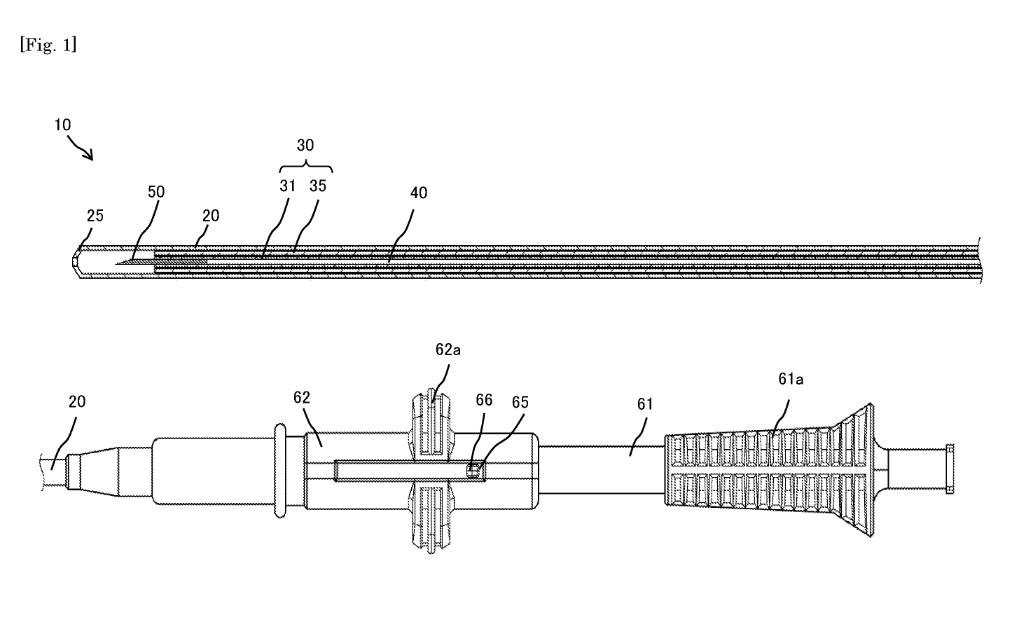

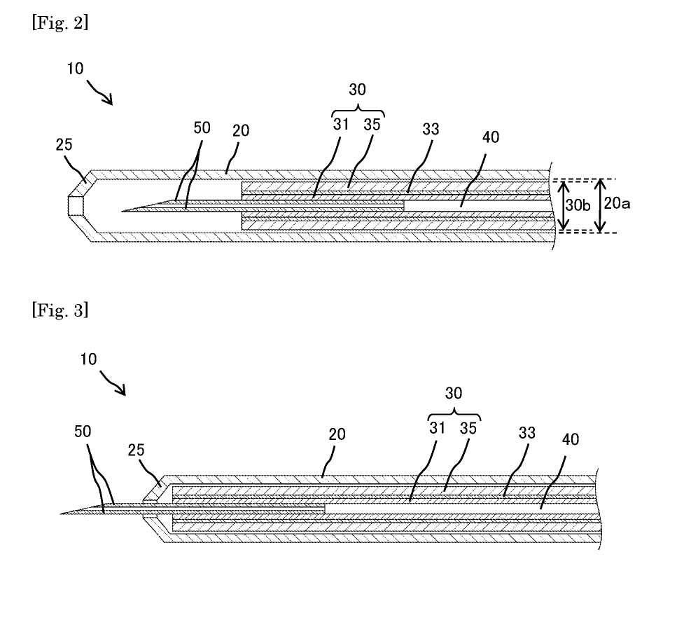

FIG. 1 shows a plan view (a partial cross-sectional view) of an injection needle device for endoscope 10 in accordance with an embodiment of the present invention, FIG. 2 shows a cross-sectional view taken along an axial direction when an injection needle 50 is stored in an outer tubular body 20 of an injection needle device for endoscope 10 in accordance with the embodiment of the present invention and FIG. 3 shows a cross-sectional view taken along an axial direction when the injection needle 50 is exposed from the outer tubular body 20 of the injection needle device for endoscope 10 in accordance with the embodiment of the present invention. In the injection needle device for endoscope 10, the inner tubular body 30 is provided in the outer tubular body 20 and the injection needle 50 is inserted into one end part of the inner tubular body 30 by thermocompression bonding. The inner tubular body 30 includes an inner layer 31 and an outer layer 35, and the inner layer 31 and the outer layer 35 are arranged in this order in a direction in which the diameter increases from a flow path 40 through which drug solution passes. In addition, the inner layer 31 is directly or indirectly contacted with the outer layer 35 through an adhesion layer 33. A reducing diameter part 25 in which an inner diameter 20a of the outer tubular body 20 is smaller than an outer diameter 30b of the inner tubular body 30 is provided at an end part of the injection needle side of the outer tubular body 20.

In the injection needle device for endoscope 10 as shown FIG. 1, a first handle part 61 capable of moving in the axial direction of the inner tubular body 30 is provided at the other end of the inner tubular body 30 (a side opposite to the end part of the injection needle side). An operator holds the first handle part 61 by his/her hand and moves it in the axial direction, so that the inner tubular body 30 and the injection needle 50 are exposed from the outer tubular body 20 or are stored in the outer tubular body 20. Although not shown, the inner tubular body 30 and the first handle part 61 are connected with each other inside the first handle part 61, the side opposite to the end part of the injection needle side of the first handle part 61 is connected with a container stored drug solution. The drug solution storage container is, for example, an injector that includes a syringe and a plunger. The inner tubular body 30 and the first handle part 61 are connected with each other by thermocompression bonding, adhesive and the like. It is also preferred to use a transparent material for the first handle part 61 so that a connection state between the inner tubular body 30 and the first handle part 61 can be confirmed.

In addition, in FIG. 1, a second handle part 62 is provided at the side opposite to the end part of the injection needle side of the outer tubular body 20. The second handle part 62 is provided outside the first handle part 61 and the first handle part 61 is capable of moving in the axial direction with respect to the second handle part 62. When the first handle part 61 and the second handle part 62 are disposed in this manner, an occurrence of a gap between the axial direction of the inner tubular body 30 and the axial direction the outer tubular body 20 can be prevented. Here, the outer tubular body 20 and the second handle part 62 can be connected with each other by thermocompression bonding, adhesive and the like as with the connection between the inner tubular body 30 and the first handle part 61.

The first handle part 61 and the second handle part 62 are composed of polymer materials such as polypropylene. The first handle part 61 and the second handle part 62 may be cylindrical or square tubular, and irregularities or slip resistance may be provided on the side faces of the handle parts to enable stable gripping. The first handle part 61 and the second handle part 62 which are cylindrical in FIG. 1, each of the first handle part 61 and the second handle part 62 is provided with projections 61a and 62a on the outer surfaces thereof on the side of the drug solution storage container for enabling easy gripping.

It is also preferred that a second handle part 62 is provided with a fixing means 65 that secures the position in the axial direction of the first handle part 61. In FIG. 1, the fixing means 65 is an opening formed on the side part of the second handle part 62. Due to the engagement between this opening and a projection 66 formed on the outer surface of the first handle part 61, the first handle part 61 and the second handle part 62 are secured to each other. When the first handle part 61 is moved toward the injection needle 50 side of the inner tubular body 30, and then, the first handle part 61 and the second handle part 62 are secured to each other with the injection needle 50 being exposed, the position of the injection needle 50 in the axial direction of the inner tubular body 30 is not changed with respect to the outer tubular body 20, whereby the needle insertion and the injection of drug solution can be performed with stability. The fixing means 65 for securing the first handle part 61 is not limited to the mode described above. For example, when the first handle part 61 is disposed on an inner side of the second handle part 62, the fixing means 65 may be a recess which is formed on an inner surface of the second handle part 62 to be engaged with the projection 66 formed on the outer surface of the first handle part 61.

The operation of the injection needle device for endoscope 10 according to the embodiment of the present invention will be described with reference to FIGS. 2 and 3. As the material for the tubular bodies in the present embodiment, nylon is used for the inner layer 31 of the inner tubular body 30, polyethylene is used for the outer layer 35, and PTFE is used for the outer tubular body 20.

As illustrated in FIG. 2, the operator firstly confirms that the inner tubular body 30 and the injection needle 50 are stored in the outer tubular body 20 before using the injection needle device for endoscope 10. Then, the operator inserts the injection needle device for endoscope 10 from a forceps opening of an endoscope, and moves the end parts of the injection needle side of the outer tubular body 20 and the inner tubular body 30 near a target tissue site of a patient.

While confirming the position of the tissue site to which the injection needle is to be inserted by use of the endoscope, the operator moves the first handle part 61 in the axial direction of the inner tubular body 30 to expose the injection needle 50, which is inserted into the inner tubular body 30, from the outer tubular body 20 as illustrated in FIG. 3, and inserts the injection needle 50 into the target tissue site of the patient. At that time, the fixing means 65 for securing the first handle part 61 is provided on the second handle part 62, and therefore, the position of the injection needle 50 in the axial direction of the inner tubular body 30 is hardly shifted with respect to the outer tubular body 20.

In the state in which the injection needle 50 is inserted into the target tissue site of the patient, an injector into which drug solution is sealed is connected to the rear of the first handle part 61, for example. When a plunger is pushed against a syringe of the injector toward the first handle part 61, drug solution is supplied into the first handle part 61, and injected into the target tissue site of the patient through the flow path 40 in the inner tubular body 30 and the injection needle 50. The reducing diameter part 25 in which the inner diameter 20a of the outer tubular body 20 is smaller than the outer diameter 30b of the inner tubular body 30 is formed on the end part of the injection needle side of the outer tubular body 20. Therefore, even when the injection needle 50 is exposed from the outer tubular body 20 by the movement of the first handle part 61 in the axial direction, the inner tubular body 30 is not exposed and keeps on being stored in the outer tubular body 20.

This application claims the benefit of the priority date of Japanese patent application No. 2014-192891 filed on Sep. 22, 2014. All of the contents of the Japanese patent application No. 2014-192891 filed on Sep. 22, 2014, are incorporated by reference herein.

### Examples

The present invention will be more specifically explained below with reference to specific examples; however, the present invention is not restricted by the below examples and can be put into practice after appropriate modifications within a range meeting the gist of the above and the below, all of which are included in the technical scope of the present invention.

A test will be described below, in which an amount of change of a position of the tip of an injection needle with respect to an outer tubular body is measured before and after an injection needle device for endoscope is left under a high humidity environment. Firstly, injection needle devices for endoscope needed for the measurement were manufactured, each of the injection needle devices for endoscope including an outer tubular body, an inner tubular body provided in the outer tubular body and having an inner layer and an outer layer, and an injection needle inserted into one end of the inner tubular body. The lengths in the axial direction of the outer tubular body and the inner tubular body were 2500 mm, the outer diameter of the outer tubular body was 2.5 mm, the inner diameter thereof was 1.9 mm, the outer diameter of the inner tubular body was 1.8 mm, and the inner diameter thereof was 1.2 mm. The outer tubular body is composed of Polypropylene (PP), and the inner tubular body is composed of nylon. The tip of the injection needle with respect to the outer tubular body after each of these injection needle devices for endoscope was left under an environment having relative humidity of 40 to 60% for 3 hours was located on the position distant from the end part (distal end part) of the injection needle side of the outer tubular body by 5 mm toward the proximal side. In this test, the position of the tip of the injection needle with respect to the outer tubular body after each of the injection needle devices for endoscope was left for 3 hours in an oven having relative humidity of 80% was measured. Notably, in this test, the material of the outer layer of the inner tubular body, the thickness of the inner layer of the inner tubular body, and the thickness of the outer layer of the inner tubular body were varied with the thickness of the inner tubular body being fixed to 300 μm and the thickness of an adhesion layer for the adhesion between the inner layer and the outer layer of the inner tubular body being fixed to 5 μm.

In Examples 1 to 3, the outer layer of the inner tubular body is composed of HDPE. In Example 1, the thickness of the inner layer is 75 the thickness of the outer layer is 220 in Example 2, the thickness of the inner layer is 30 the thickness of the outer layer is 265 and in Example 3, the thickness of the inner layer is 100 the thickness of the outer layer is 195 μm.

In Examples 4 and 5, the outer layer of the inner tubular body is composed of PTFE. In Example 4, the thickness of the inner layer is 30 the thickness of the outer layer is 265 and in Example 5, the thickness of the inner layer is 100 the thickness of the outer layer is 195

In Example 6, the outer layer of the inner tubular body is composed of ETFE. In Example 6, the thickness of the inner layer is 30 the thickness of the outer layer is 265 μm.

Table 2 shows: a humidity when the injection needle device for endoscope is left for under an high humidity environment (%); a humidification time (hour); an outer diameter (mm), an inner diameter (mm), a material and a length in an axial direction before and after a humidification (mm) of the outer tubular body; an outer diameter (mm), an inner diameter (mm), a material of the inner layer, a thickness d1 of the inner layer (μm), a material of the outer layer, a thickness d2 of the outer layer (μm), a thickness ratio (d2/d1) of the outer layer to the inner layer and a length in an axial direction before and after the humidification (mm) of the inner tubular body; a state of the injection needle after the humidification.

TABLE 2

| | | | Outer tubular body | | | | | | |
|---|---|---|---|---|---|---|---|---|---|
| | | | | | | Length in axial direction | | Inner tubular body | |
| | | Humidification | Outer | Inner | | (mm) | | Outer | Inner |
| | Humidity | time | diameter | diameter | | Before | After | diameter | diameter |
| | (%) | (hour) | (mm) | (mm) | Material | humidification | humidification | (mm) | (mm) |
| Example 1 | 80 | 3 | 2.5 | 1.9 | PP | 2500 | 2500 | 1.8 | 1.2 |
| Example 2 | 80 | 3 | 2.5 | 1.9 | PP | 2500 | 2500 | 1.8 | 1.2 |
| Example 3 | 80 | 3 | 2.5 | 1.9 | PP | 2500 | 2500 | 1.8 | 1.2 |
| Example 4 | 80 | 3 | 2.5 | 1.9 | PP | 2500 | 2500 | 1.8 | 1.2 |
| Example 5 | 80 | 3 | 2.5 | 1.9 | PP | 2500 | 2500 | 1.8 | 1.2 |
| Example 6 | 80 | 3 | 2.5 | 1.9 | PP | 2500 | 2500 | 1.8 | 1.2 |

| | Inner tubular body | | | | | | | |
|---|---|---|---|---|---|---|---|---|
| | Inner layer | | Outer layer | | | Length in axial direction | | State of |
| | | Thickness | | Thickness | Thickness | (mm) | | injection |
| | | d1 | | d2 | ratio | Before | After | needle after |
| | Material | (μm) | Material | (μm) | (d2/d1) | humidification | humidification | humidification |
| Example 1 | Nylon | 75 | HDPE | 220 | 2.93 | 2500 | 2503 | Stored |
| Example 2 | Nylon | 30 | HDPE | 265 | 8.83 | 2500 | 2500 | Stored |
| Example 3 | Nylon | 100 | HDPE | 195 | 1.95 | 2500 | 2506 | Partly |
| | | | | | | | | exposed |
| Example 4 | Nylon | 30 | PTFE | 265 | 8.83 | 2500 | 2500 | Stored |
| Example 5 | Nylon | 100 | PTFE | 195 | 1.95 | 2500 | 2506 | Partly |
| | | | | | | | | exposed |
| Example 6 | Nylon | 30 | ETFE | 265 | 8.83 | 2500 | 2500 | Stored |

### Examples 1 to 3

In Example 1, after the humidification, the tip of the injection needle was located on the position distant from the end part of the outer tubular body by 2 mm toward the proximal side, and the length in the axial direction of the inner tubular body was 2503 mm. However, the injection needle was not exposed from the outer tubular body. In Example 2, even after the humidification, the tip of the injection needle was still located on the position distant from the end part of the outer tubular body by 5 mm toward the proximal side, and this indicates that the length in the axial direction of the inner tubular body was not changed from 2500 mm. In addition, the injection needle was not exposed from the outer tubular body. In Example 3, after the humidification, the tip of the injection needle was located on the position distant from the distal end part of the outer tubular body by 1 mm toward the distal side, which indicates that the length in the axial direction of the inner tubular body was 2506 mm. Although Example 3 provides a certain effect of suppressing the elongation percentage (100×((length after being left under high humidity environment/length before being left under high humidity environment)−1)) of the inner tubular body in the axial direction to about 0.24%, the injection needle was exposed from the outer tubular body by about 1 mm.

### Examples 4 and 5

In Example 4, even after the humidification, the tip of the injection needle was still located on the position distant from the end part of the outer tubular body by 5 mm toward the proximal side, and this indicates that the length in the axial direction of the inner tubular body was not changed from 2500 mm. In addition, the injection needle was not exposed from the outer tubular body. On the other hand, in Example 5, after the humidification, the tip of the injection needle was located on the position distant from the end part of the outer tubular body by 1 mm toward the distal side, and the length in the axial direction of the inner tubular body was 2506 mm. Although Example 5 provides a certain effect of suppressing the elongation percentage of the inner tubular body in the axial direction to about 0.24%, the injection needle was exposed from the outer tubular body by about 1 mm.

### Example 6

In Example 6, even after the humidification, the length in the axial direction of the inner layer was not changed from 2500 mm, and the injection needle was not exposed from the outer tubular body.

Note that, even under any of the conditions in Examples 1 to 6, the length in the axial direction of the outer tubular body which had been 2500 mm before the humidification still remained at 2500 mm after the humidification.

It is found from the test result described above that, according to the configuration in which an inner tubular body of an injection needle device for endoscope has a multi-layer structure including at least an inner layer and an outer layer and a water absorption coefficient of the outer layer is set to be lower than a water absorption coefficient of the inner layer, the inner tubular body that hardly expands even under a high humidity environment can be manufactured. In addition, the thickness of the inner layer may be smaller than the thickness of the outer layer. For example, the thickness of the outer layer is preferably 2.5 times or more of that of the inner layer.

### REFERENCE SIGNS LIST

10: an injection needle device
20: an outer tubular body
25: a reducing diameter part
30: an inner tubular body
31: an inner layer
33: an adhesion layer
35: an outer layer
50: an injection needle
61: a first handle part
62: a second handle part
65: a fixing means

1. An injection needle device for an endoscope comprising:
an outer tubular body;
an inner tubular body provided in the outer tubular body; and
an injection needle inserted into one end part of the inner tubular body; wherein
the inner tubular body includes an inner layer and an outer layer directly or indirectly contacted with an outer surface of the inner layer, and
a water absorption coefficient of the outer layer is lower than that of the inner layer.

2. The injection needle device for an endoscope according to claim 1, wherein
the water absorption coefficient of the outer layer is 0.04 times or less of that of the inner layer.

3. The injection needle device for an endoscope according to claim 1, wherein
a thickness of the outer layer is 2.5 times or more of that of the inner layer.

4. The injection needle device for an endoscope according to claim 1, wherein
a length in an axial direction of the inner tubular body after being left under the following high humidity environment is 1.013 times or less of that before being left under the following high humidity environment, when the inner tubular body is left for three hours under an environment of relative humidity of 40% to 60% and subsequently left for three hours under a high humidity environment of relative humidity of 80% or more.

5. The injection needle device for an endoscope according to claim 1, wherein
a contact angle of the inner layer is smaller than that of the outer layer.

6. The injection needle device for an endoscope according to claim 1, wherein
the inner layer is composed of nylon, and the outer layer is composed of polyethylene.

7. The injection needle device for an endoscope according to claim 1, wherein
the injection needle and the inner layer are connected with each other by thermocompression bonding.

8. The injection needle device for an endoscope according to claim 1, further comprising
a first handle part connected to the other end of the inner tubular body and capable of moving in the axial direction of the inner tubular body.

9. The injection needle device for an endoscope according to claim 8, further comprising
a second handle part connected to the outer tubular body and provided with a fixing means that secures the first handle part.

10. The injection needle device for an endoscope according to claim 1, wherein
an inner diameter at an end part of the injection needle side of the outer tubular body is smaller than an outer diameter of the inner tubular body.

11. The injection needle device for an endoscope according to claim 1, wherein
a length of the inner tubular body is longer than that of the outer tubular body in the axial direction of the inner tubular body.

12. The injection needle device for an endoscope according to claim 1, wherein
the outer tubular body is composed of polytetrafluoroethylene, and the outer layer of the inner tubular body is composed of a material other than polytetrafluoroethylene.
